(19) United States
(12) Patent Application Publication
(10) Pub. No.: US 2023/0035236 A1
(43) Pub. Date: Feb. 2, 2023

(54) SOFTENING BASE AGENT

(71) Applicant: KAO CORPORATION, Tokyo (JP)

(72) Inventors: Takako IGARASHI, Wakayama-shi, Wakayama (JP); Ojiro TAMAGAWA, Wakayama-shi, Wakayama (JP)

(73) Assignee: KAO CORPORATION, Tokyo (JP)

(21) Appl. No.: 17/783,689

(22) PCT Filed: Dec. 18, 2020
(86) PCT No.: PCT/JP2020/047449

§ 371 Date: Jun. 9, 2022

(30) Foreign Application Priority Data
Dec. 20, 2019 (JP) 2019-229980

(51) Int. Cl. C11D 3/34; C11D 11/00; C11D 3/00; C07C 309/17

(52) U.S. Cl. CPC C11D 3/3472; C11D 11/0017; C11D 3/0015; C07C 309/17

(57) ABSTRACT

The present invention is a softening base agent containing a compound represented by the following formula 1:

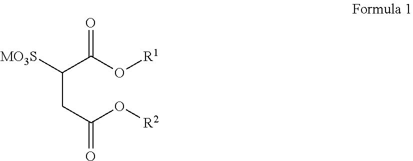

wherein each of R¹ and R² represents a hydrocarbon group with 6 or more and 24 or less carbons, and the total number of carbons in R¹ and R² is 18 or more and 30 or less; and M is a cation, excluding a hydrogen ion.

### FIELD OF THE INVENTION

The present invention relates to a softening base agent, a fiber treatment agent composition and a method for treating fibers.

### BACKGROUND OF THE INVENTION

Textile products such as clothing or the like gradually become stiff and undesirable in textures as they are repeatedly worn or washed. They are softened with fabric softeners added in rinsing processes during washing or the like to improve this.

While most softening agent compositions commercially available at present contain cationic surfactants as active ingredients, softening agent compositions containing anionic surfactants have been conventionally studied. For example, JP-A 2005-171399 discloses a softening agent composition consisting of a neutralized or quaternized product of an amine compound and a sulfonate-type anionic surfactant, the composition imparting excellent softness to various types of textile products without impairing the water absorbency of hydrophilic textile products. Further, JP-A H8-158258 discloses an antibacterial softening agent composition comprising a cationic bactericidal agent such as benzalkonium chloride or the like, an α-olefin sulfonate salt and/or a dialkyl sulfosuccinate salt as essential components, the composition imparting excellent textures to various types of fibers and exhibiting an excellent antibacterial effect. In addition, U.S. Pat. No. 5,419,842 discloses a fabric softening aqueous emulsion comprising a higher fatty acid ester of pentaerythritol, an oligomer of pentaerythritol, a lower alkylene oxide derivative of pentaerythritol, a lower alkylene oxide derivative of an oligomer of pentaerythritol or a mixture of any two or more thereof as a fabric softening compound, the emulsion further comprising an anionic emulsifier selected from the group consisting of diisotridecyl sulfosuccinate, diisodecyl sulfosuccinate and an alkali metal salt of a fatty acid, an ethoxylated alcohol emulsifier and an aqueous medium in amounts falling within their respective predetermined ranges, wherein the emulsion is essentially free of cationic emulsifiers and fabric softeners. This literature states that a predetermined compound such as a higher fatty acid ester of pentaerythritol or the like is the only fabric softening compound other than bentonite.

### SUMMARY OF THE INVENTION

While anionic surfactants are preferable components from the viewpoint of formulation as softening agent compositions using them cause less discoloration, softening base agents using anionic surfactants are desired to have further improved softening effects.

Further, fiber treatment agent compositions such as softening agent compositions or the like are generally formulated in the form of dispersions including water and desired to be stable in such forms.

The present invention provides a softening base agent using an anionic surfactant, the softening base agent having excellent dispersion stability in formulations and being superior to other anionic surfactants in fiber softening effect.

The present invention relates to a softening base agent containing a compound represented by the following formula 1 (hereinafter referred to as compound 1):

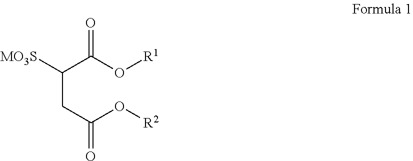

wherein each of R¹ and R² represents a hydrocarbon group with 6 or more and 24 or less carbons, and the total number of carbons in R¹ and R² is 18 or more and 30 or less; and M is a cation, excluding a hydrogen ion.

Further, the present invention relates to a fiber treatment agent composition containing the softening base agent of the present invention.

Further, the present invention relates to a method for treating fibers including, treating the fibers with the softening base agent of the present invention, wherein the compound represented by the formula 1 is used in an amount of 0.01% o.w.f. or more and 5% o.w.f. or less relative to the fibers.

According to the present invention, provided is a softening base agent using an anionic surfactant, the softening base agent having excellent dispersion stability in formulations and being superior to other anionic surfactants in fiber softening effect.

### EMBODIMENTS OF THE INVENTION

### [Softening Base Agent]

The softening base agent of the present invention means an agent (active ingredient) that develops softness to fibers.

The present invention relates to a softening base agent containing compound 1 represented by the formula 1. The softening base agent of the present invention may be composed of compound 1. Further, the softening base agent of the present invention may contain one or more types of compound 1 or may be composed of one or more types of compound 1.

Compound 1 is a sulfosuccinate salt with two hydrocarbon groups having specific structures, and is excellent in dispersion stability in aqueous systems and capable of imparting excellent softness to fibers. The reason why compound 1 exhibits such effects is not clear, but it is considered as follows.

Generally, anionic surfactants of the sulfonate-type or the like having long alkyl chains are high in Krafft point and likely to be flocculated and separated in water. Uniform treatment of fibers with flocculating dispersions is difficult even if carrying out bathing treatment against them, so that performance is less likely to be developed. On the other hand, in the present invention, specific structures and specific hydrocarbon groups (for example, carbon numbers of alkyl groups and structures of alkyl groups) of compound 1 represented by the formula 1 optimize hydrophilicity/hydrophobicity, flocculating properties and curvature, and enable the preparation of stable water dispersions. It is inferred that uniform and efficient treatment of fibers can be carried out in such a stable dispersion state, resulting in the development of excellent fiber modifying effects. Note that the present invention is not restrained by this mechanism.

In the formula 1, R¹ and R² may be the same or different, and each of them is a hydrocarbon group with 6 or more and 24 or less carbons. Examples of the hydrocarbon group include an alkyl group and an alkenyl group.

In the formula 1, each of the hydrocarbon groups of R¹ and R² has 6 or more, preferably 8 or more and more preferably 10 or more carbons from the viewpoint of softness, and 24 or less, preferably 20 or less and more preferably 17 or less carbons from the viewpoint of dispersibility.

In the formula 1, the total number of carbons in R¹ and R² is 18 or more, preferably 20 or more, more preferably 21 or more and further preferably 22 or more from the viewpoint of softness, and 30 or less, preferably 28 or less, more preferably 26 or less and further preferably 25 or less from the viewpoint of dispersibility. Here, when the softening base agent contains two or more compounds having different total numbers of carbons in R¹ and R², the total number of carbons in R¹ and R² in the softening base agent represents the molar average of the total numbers of carbons in R¹ and R² of those compounds.

In the formula 1, the hydrocarbon groups of R¹ and R² may be either linear or branched, but preferably include a branched one from the viewpoint of dispersibility. In other words, the hydrocarbon groups of R¹ and R² preferably include a hydrocarbon group having a branched structure.

In the formula 1, the hydrocarbon groups of R¹ and R² may be either saturated or unsaturated, but preferably include an unsaturated one from the viewpoint of dispersibility. In other words, the hydrocarbon groups of R¹ and R² preferably include a hydrocarbon group having an unsaturated bond.

Accordingly, at least one of R¹ and R² in the formula 1 is preferably a hydrocarbon group having a branched structure or an unsaturated bond.

In the formula 1, the hydrocarbon groups of R¹ and R² more preferably include a saturated branched one or an unsaturated linear one from the viewpoint of dispersibility. Each of the hydrocarbon groups of R¹ and R² is preferably a saturated branched one from the viewpoints of dispersion stability, and softness at a high hardness (for example, 20° DH), and preferably includes an unsaturated linear one from the viewpoints of dispersion stability, and softness at a medium hardness (for example, 8° DH).

In the formula 1, the hydrocarbon groups of R¹ and R² may be the same or different. The hydrocarbon groups of R¹ and R² are preferably different from the viewpoint of softness at a high hardness. Further, the hydrocarbon groups of R¹ and R² are preferably the same from the viewpoints of dispersion stability, ease of production and softness in a low concentration. For example, R¹ and R² in the formula 1 may have the same or different numbers of carbons. R¹ and R² preferably have different numbers of carbons from the viewpoint of softness at a high hardness. Further, R¹ and R² preferably have the same number of carbons from the viewpoints of dispersion stability, ease of production and softness in a low concentration.

When the hydrocarbon groups of R¹ and R² in the formula 1 include a hydrocarbon group having a branched structure, the hydrocarbon groups of R¹ and R² each has preferably 1 or more and 2 or less, more preferably 1 or more and 1.5 or less, further preferably 1 or more and 1.2 or less, furthermore preferably 1 or more and 1.1 or less and furthermore preferably 1 branch from the viewpoints of softness and dispersion stability. Here, the number of branches is expressed by the number average of the numbers of branches in hydrocarbon groups having branched structures. Each of R¹ and R² preferably has 1 branch.

Each of the hydrocarbon groups of R¹ and R² having branched structures is preferably a hydrocarbon group having a branched chain in the second position, more preferably a hydrocarbon group having a branched chain with 2 or more carbons in the second position, further preferably an alkyl group having a branched chain with 2 or more carbons in the second position, furthermore preferably a hydrocarbon group having a branched chain derived from a Guerbet alcohol in the second position, and furthermore preferably a hydrocarbon group having a branched chain derived from a Guerbet alcohol only in the second position from the viewpoints of softness and dispersibility.

Note that, in the present invention, with the carbon of R¹ or R² bonded to O of —O—R¹ or —O—R² in the formula 1 defined as a carbon in the first position, a carbon bonded to the carbon in the first position is defined as a carbon in the second position, a carbon bonded to the carbon in the second position is defined as a carbon in the third position and a carbon bonded to the carbon in the third position is defined as a carbon in the fourth position, and the same applies to carbons in the fifth, sixth or subsequent positions. That is, the position of a carbon in R¹ and R² is determined by selecting the longest main chain including the carbon in the first position and determining the position of the carbon such as the second or subsequent positions on the basis of the main chain.

When the hydrocarbon groups of R¹ and R² in the formula 1 include a hydrocarbon group having a branched structure, the proportion of a hydrocarbon group having a branched structure at a carbon in the second or subsequent positions and only one methyl group bonded to the carbon in the second position (hereinafter also referred to as hydrocarbon group B2 of R¹ and R²) is preferably 5 mol % or less, more preferably 4 mol % or less, further preferably 3 mol % or less, furthermore preferably 2 mol % or less, furthermore preferably 1 mol % or less and furthermore preferably 0 mol % in all the hydrocarbon groups of R¹ and R² from the viewpoint of softness.

Hydrocarbon group B2 of R¹ and R² is a hydrocarbon group represented by the formula 2:

—CH₂—CH(CH₃)—R²¹ Formula 2

wherein R²¹ is a hydrocarbon group with 3 or more and 21 or less carbons.

R²¹ is preferably an alkyl group, more preferably a linear alkyl group and further preferably a primary linear alkyl group from the viewpoint of availability.

R²¹ in the formula 3 has preferably 5 or more and more preferably 6 or more carbons from the viewpoint of softness, and preferably 17 or less and more preferably 14 or less carbons from the viewpoint of dispersibility.

When the hydrocarbon groups of R¹ and R² in the formula 1 include a hydrocarbon group having a branched structure, the proportion of a hydrocarbon group having a branched structure only at a carbon in the third or subsequent positions and only one methyl group bonded to the carbon in the third position (hereinafter also referred to as hydrocarbon group B3 of R¹ and R²) is preferably 10 mol % or less, more preferably 5 mol % or less, further preferably 2 mol % or less, furthermore preferably 1 mol % or less and furthermore preferably 0 mol % in all the hydrocarbon groups of R¹ and R² from the viewpoint of softness.

Hydrocarbon group B3 of R¹ and R² is a hydrocarbon group represented by the formula 3:

—R³¹—CH(CH₃)—R³² Formula 3

wherein R³¹ is a linear hydrocarbon group with 2 carbons, R³² is a hydrocarbon group, and the total number of carbons in R³¹ and R³² is 4 or more and 22 or less.

R³¹ is preferably an ethanediyl group from the viewpoint of availability, and R⁴ is preferably an alkyl group, more preferably a linear alkyl group and further preferably a primary linear alkyl group from the viewpoint of availability.

The total number of carbons in R³¹ and R³² in the formula 3 is 4 or more, preferably 6 or more and more preferably 8 or more from the viewpoint of softness, and 22 or less, preferably 18 or less and more preferably 15 or less from the viewpoint of dispersibility.

When the hydrocarbon groups of R¹ and R² in the formula 1 include a hydrocarbon group having a branched structure, the proportion of a hydrocarbon group having a branched structure only at a carbon in the fourth or subsequent positions and only one methyl group bonded to the carbon at the smallest branch position (hereinafter also referred to as hydrocarbon group B4 of R¹ and R²) is preferably 50 mol % or less, more preferably 40 mol % or less, further preferably 30 mol % or less, furthermore preferably 20 mol % or less, furthermore preferably 10 mol % or less, furthermore preferably 5 mol % or less, furthermore preferably 1 mol % or less and furthermore preferably 0 mol % in all the hydrocarbon groups of R¹ and R² from the viewpoint of softness.

Hydrocarbon group B4 of R¹ and R² is a hydrocarbon group represented by the formula 4:

—R⁴¹—CH(CH₃)—R⁴² Formula 4

wherein R⁴¹ is a linear hydrocarbon group with 3 or more carbons, R⁴² is a hydrocarbon group, and the total number of carbons in R⁴¹ and R⁴² is 4 or more and 22 or less.

R⁴¹ is preferably an alkane-α,ω-diyl group with 3 or more carbons from the viewpoint of availability, R⁴² is preferably an alkyl group, more preferably a linear alkyl group and further preferably a primary saturated linear alkyl group from the viewpoint of availability.

The total number of carbons in R⁴¹ and R⁴² in the formula 4 is 4 or more, preferably 6 or more and more preferably 8 or more from the viewpoint of softness, and 22 or less, preferably 18 or less and more preferably 15 or less from the viewpoint of dispersibility.

The proportion of the hydrocarbon group having a branched structure at a carbon in the second or subsequent positions and only one methyl group bonded to the carbon in the second position, the proportion of the hydrocarbon group having a branched structure only at a carbon in the third or subsequent positions and only one methyl group bonded to the carbon in the third position, the proportion of the hydrocarbon group having a branched structure only at a carbon in the fourth or subsequent positions and only one methyl group bonded to the carbon at the smallest branch position or the like relative to all the hydrocarbon groups of R¹ and R² can each be measured by ¹³C-NMR after converting —O—R¹ and —O—R² respectively to alcohols H—O—R¹ and H—O—R² through hydrolysis or the like of the compound represented by the formula 1.

In results of the measurement by ¹³C-NMR under the conditions below, compound 1 has a proportion of the area of the region of signals in the range of 67.6 to 68 ppm relative to the area of the region of all signals between 60 and 69 ppm of preferably 5% or less, more preferably 4% or less, further preferably 3% or less, furthermore preferably 2%, or less, furthermore preferably 1% or less and furthermore preferably 0%. Note that, in the following ¹³C-NMR, a chemical shift of the carbon in the first position can be determined from a plurality of branched alcohols having methyl branches as standard materials and utilized to predict chemical shifts in a sample.

### ¹³C-NMR Measurement Conditions

Equipment: manufactured by Agilent Technologies, MR 400

Frequency: 400 MHz

Cumulative number: 1024

Latency time: 30 sec

Pulse angle: 45 deg

Deuterated solvent: CDCl₃

Sample concentration: 10%

Sample tube: 5 mmφ

In results of the measurement by ¹³C-NMR under the above conditions, compound 1 has a proportion of the area of the region of signals in the range of 60 to 61 ppm relative to the area of the region of all signals between 60 and 69 ppm of preferably 10% or less, more preferably 5% or less, further preferably 2% or less, furthermore preferably 1% or less and furthermore preferably 0%.

In results of the measurement by ¹³C-NMR under the above conditions, compound 1 has a proportion of the area of the region of signals in the range of 62 to 63.2 ppm relative to the area of the region of all signals between 60 and 69 ppm of preferably 50% or less, more preferably 40% or less, further preferably 30% or less, furthermore preferably 20% or less, furthermore preferably 10% or less, furthermore preferably 5% or less, furthermore preferably 1%, or less and furthermore preferably 0%.

When the hydrocarbon groups of R¹ and R² in the formula 1 include a hydrocarbon group having a branched structure, the degree of branching of R¹ and R² that is defined by the following formula is preferably 0.3 or less, more preferably 0.2 or less and further preferably 0.1 or less from the viewpoint of softness, and preferably 0.01 or more, more preferably 0.02 or more and further preferably 0.04 or more from the viewpoint of dispersibility.

degree of branching=[(total number of terminal methyl groups in R¹ and R²)−2]/(total number of carbons in R¹ and R²)

Note that, as the numbers of carbons used to calculate the degree of branching, average values of those measured by ¹H-NMR can be used.

When the hydrocarbon groups of R¹ and R² in the formula 1 include a hydrocarbon group having an unsaturated bond, the unsaturated bond in the hydrocarbon groups of R¹ and R² is preferably a carbon-carbon double bond from the viewpoints of softness and dispersion stability.

When the hydrocarbon groups of R¹ and R² in the formula 1 include a hydrocarbon group having an unsaturated bond, the number of unsaturated bonds in the hydrocarbon groups of R¹ and R² is preferably 0.5 or more and 2 or less, more preferably 1 or more and 1.5 or less, further preferably 1 or more and 1.2 or less, furthermore preferably 1 or more and 1.1 or less, and furthermore preferably 1 from the viewpoints of softness, dispersion stability and availability. Here, the number of unsaturated bonds is expressed by the number average of the numbers of unsaturated bonds in hydrocarbon groups having unsaturated bonds.

When the hydrocarbon groups of R¹ and R² in the formula 1 include a hydrocarbon group having a double bond, the suitable range of the number of double bonds in the hydrocarbon groups of R¹ and R² is that of the number of unsaturated bonds.

Compound 1 or the compound represented by the formula 1 may be one or more compounds selected from a compound in which R¹ and R² are hydrocarbon groups having the same structure, and a compound in which R¹ and R² are hydrocarbon groups having different structures.

Compound 1 is preferably a compound in which R¹ and R² are hydrocarbon groups having different structures from the viewpoint of softness.

For example, the softening base agent of the present invention can contain a compound represented by the formula 1 in which R¹ and R² are hydrocarbon groups having the same structure, and a compound represented by the formula 1 in which R¹ and R² are hydrocarbon groups having different structures. The softening base agent of the present invention may contain as compound 1 the compound in which R¹ and R² are hydrocarbon groups having different structures.

M in the formula 1 is a cation, excluding a hydrogen ion. Examples of M include, for example, alkali metal ions such as a lithium ion, a sodium ion, a potassium ion and the like, alkaline earth metal ions such as a calcium ion, a barium ion and the like, organic ammonium ions such as a triethanolammonium ion, a diethanolammonium ion, a monoethanolammonium ion, a trimethylammonium ion, a monomethylammonium ion and the like, and others.

M is preferably an alkali metal ion or an alkanolammonium ion, more preferably a sodium ion, a potassium ion, a triethanolammonium ion, a diethanolammonium ion or a monoethanolammonium ion, and further preferably a sodium ion from the viewpoints of dispersion stability and softness.

Compound 1 can be synthesized by a publicly-known method. For example, a maleic acid diester obtained by reacting an alcohol with a maleic anhydrite can be reacted with a hydrogen sulfite to obtain compound 1. At that time, if alcohols different in carbon numbers or structures is used, compounds in which R¹ and R² in the formula 1 are hydrocarbon groups having different structures can be obtained. Compound 1 can be synthesized, for example, by the methods described in examples 2 to 3 of US-A 2007-0214999. Examples of the alcohol include, for example, a linear alcohol and an alcohol having a branch in the second position. Examples of the alcohol having a branch in the second position include a Guerbet alcohol.

The softening base agent of the present invention can be directed to various types of fibers, for example, natural fibers, synthetic fibers and semi-synthetic fibers. Further, the softening base agent of the present invention can be directed to textile products including these fibers.

The fibers may be either hydrophobic fibers or hydrophilic fibers. Examples of hydrophobic fibers include, for example, protein fibers (milk protein casein fiber, promix and the like), polyamide fibers (nylon and the like), polyester fibers (polyester and the like), polyacrylonitrile fibers (acrylic and the like), polyvinyl alcohol fibers (vinylon and the like), polyvinyl chloride fibers (polyvinyl chloride and the like), polyvinylidene chloride fibers (vinylidene and the like), polyolefin fibers (polyethylene, polypropylene and the like), polyurethane fibers (polyurethane and the like), polyvinyl chloride/polyvinyl alcohol copolymer fibers (polychlal and the like), polyalkylene paraoxybenzoate fibers (benzoate and the like), polyfluoroethylene fibers (polytetrafluoroethylene and the like) and others. Examples of hydrophilic fibers include, for example, seed hair fibers (cotton, cotton, kapok and the like), bast fibers (hemp, flax, ramie, cannabis, jute and the like), vein fibers (manila hemp, sisal hemp and the like), palm fibers, juncus, straw, animal hair fibers (wool, mohair, cashmere, camel hair, alpaca, vicuna, angora and the like), silk fibers (domestic silk and wild silk), feathers, cellulose fibers (rayon, polynosic, cupro, acetate and the like) and others.

The fibers are preferably fibers including cotton fibers. The content of cotton fibers in the fibers is preferably 5 mass % or more, more preferably 10 mass % or more, further preferably 15 mass, or more, furthermore preferably 20 mass, or more and furthermore preferably 100 mass % from the viewpoint of further improving the softness of the fibers.

In the present invention, textile products mean woven fabrics, knitted fabrics, nonwoven fabrics and other fabrics using the above hydrophobic or hydrophilic fibers, and undershirts, T-shirts, dress shirts, blouses, slacks, caps, handkerchiefs, towels, knitwear, socks, underwear, tights and other products obtained from them. The textile products are preferably textile products including cotton fibers from the viewpoint of the effect of improving textures of fibers after treating with the fiber treatment agent composition of the present invention being more likely to be felt. Preferable modes of the content of cotton fibers in the textile products are the same as those of the content of cotton fibers in the fibers.

The softening base agent of the present invention can impart softness to fibers. Further, the softening base agent of the present invention is also excellent in dispersibility when mixed with water.

The present invention discloses the use of compound 1 represented by the formula 1 as a softening base agent. The matters mentioned in the softening base agent, the below-mentioned fiber treatment agent composition and method for treating fibers of the present invention can be appropriately applied to the use of the present invention. Specific examples or preferable modes of compound 1 or the like are also the same as those in the softening base agent of the present invention.

### [Fiber Treatment Agent Composition]

The present invention provides a fiber treatment agent composition containing the softening base agent of the present invention. The matters mentioned in the softening base agent of the present invention can be appropriately applied to the fiber treatment agent composition of the present invention. Specific examples or preferable modes of compound 1 or the like are also the same as those in the softening base agent of the present invention.

The content of compound 1 in the fiber treatment agent composition of the present invention is preferably 1 mass, or more, more preferably 2 mass % or more and further preferably 4 mass % or more from the viewpoint of transportability, and preferably 90 mass % or less, more preferably 70 mass % or less and further preferably 50 mass % or less from the viewpoint of handleability.

The fiber treatment agent composition of the present invention can also contain a softening base agent other than compound 1 (hereinafter also referred to as optional softening base agent), but the content thereof may be small. The content of the optional softening base agent in the fiber treatment agent composition of the present invention may be, for example, less than 1 mass % and further less than 0.1 mass % in the composition from the viewpoints of softness, dispersibility and water absorbency. Further, the mass ratio of (content of optional softening base agent)/(content of compound 1) in the fiber treatment agent composition of the present invention may be, for example, 4.5 or less, further 4.0 or less, further 3.0 or less, further 2.0 or less, further 1.0 or less, further 0.50 or less, further 0.30 or less, further less than 0.10 and further 0.050 or less from the same viewpoints. Here, examples of the optional softening base agent include, for example, a cationic softening agent and a nonionic softening base agent. The cationic softening base agent can be selected from, for example, quaternary ammonium salts. The nonionic softening base agent can be selected from, for example, a higher fatty acid ester of pentaerythritol, an oligomer of pentaerythritol, a lower alkylene oxide derivative of pentaerythritol, a lower alkylene oxide derivative of an oligomer of pentaerythritol and the like.

The fiber treatment agent composition of the present invention preferably contains water. The fiber treatment agent composition of the present invention is preferably a liquid composition containing water. Water is usually the balance of the composition and used in such an amount that makes the total of the composition 100 mass %.

When the fiber treatment agent composition of the present invention contains water, the proportion of compound 1 in the total of components other than water may be, for example, 20 mass % or more, further 30 mass % or more, further 40 mass, or more, further 50 mass % or more, further 60 mass % or more, further 70 mass % or more, further 80 mass % or more, further 90 mass % or more, further 92 mass % or more and further 95 mass % or more. The upper limit may be 100 mass % or less.

The fiber treatment agent composition of the present invention may have a pH at 20° C. of, for example, 4.0 or more, further 5.0 or more, further 5.5 or more, further 6.0 or more and further 7.0 or more.

The fiber treatment agent composition of the present invention may be a softening agent composition. For example, the present invention can provide a softening agent composition containing compound 1 as an active ingredient of the softening base agent.

As compound 1 has good dispersibility in water, the fiber treatment agent composition of the present invention containing water can be produced by mixing compound 1 with water at a relatively low temperature. The temperature of water to be mixed with compound 1 may be, for example, 30° C. or more and 50° C. or less. According to the present invention, provided is a method for producing a fiber treatment agent composition including, mixing compound 1 with water at 30° C. or more and 50° C. or less.

### [Method for Treating Fibers]

The present invention provides a method for treating fibers including, treating the fibers with the softening base agent of the present invention, wherein compound 1 is used in an amount of 0.01% o.w.f. or more and 5% o.w.f. or less relative to the fibers. Further, the present invention may be a method for treating fibers including, treating the fibers with the softening base agent of the present invention, wherein compound 1 is used in an amount of 0.05% o.w.f. or more and 5%, o.w.f. or less relative to the fibers. The matters mentioned in the softening base agent and the fiber treatment agent composition of the present invention can be appropriately applied to the method for treating fibers of the present invention. Specific examples or preferable modes of compound 1 or the like are also the same as those in the softening base agent of the present invention. The method for treating fibers of the present invention may be a method for treating fibers, wherein compound 1 is applied to the fibers in an amount of 0.01% o.w.f. or more and 5% o.w.f. or less to impart softness to the fibers. Further, the method for treating fibers of the present invention may be a method for treating fibers, wherein compound 1 is applied to the fibers in an amount of 0.05% o.w.f. or more and 5% o.w.f. or less to impart softness to the fibers. The fiber treatment agent composition of the present invention can be used in the method for treating fibers of the present invention.

In the present invention, compound 1 is used in an amount of 0.01% o.w.f. or more, preferably 0.05% o.w.f. or more, more preferably 0.1% o.w.f. or more, further preferably 0.2% o.w.f. or more and furthermore preferably 0.3% o.w.f. or more relative to the fibers from the viewpoint of softness, and 5% o.w.f. or less, preferably 4% o.w.f. or less, more preferably 3% o.w.f. or less and further preferably 2%, o.w.f. or less relative to the fibers from the viewpoint of textures. Note that % o.w.f. is an abbreviation of % on the weight of fabric, and means the percentage of the mass of compound 1 relative to the mass of fibers. In the present invention, a treatment liquid obtained by mixing the softening base agent of the present invention or the fiber treatment agent composition of the present invention with water can be brought into contact with the fibers. For example, the treatment liquid can be used such that the amount of compound 1 relative to the fibers falls within the above range.

In the present invention, the softening base agent is preferably used by mixing with water having a hardness of 0° DH or more and 30° DH or less. That is, the fibers are preferably treated with a treatment liquid obtained by mixing the softening base agent with water having a hardness of 0° DH or more and 30° DH or less. The hardness of water is preferably 1° DH or more, more preferably 2° DH or more and further preferably 3° DH or more from the viewpoint of softness, and preferably 25° DH or less and more preferably 20° DH or less from the viewpoint of textures.

The method for treating fibers of the present invention can be directed to the fibers mentioned in the softening base agent of the present invention. For example, the fibers may be fibers of fabric.

The method for treating fibers of the present invention can be performed by incorporating into washing processes of fibers, for example, fibers of fabric. Here, the washing processes may be treatments such as washing, rinsing and dewatering fibers. In the present invention, the softening base agent of the present invention can be applied to fibers in any of these washing processes such that compound 1 is used in a predetermined amount.

The present invention discloses, in addition to the above embodiments, the following aspects.

<1>

A softening base agent containing a compound represented by the following formula 1:

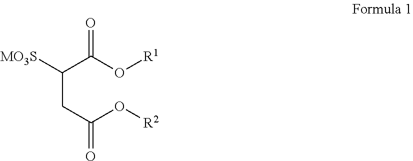

wherein each of R¹ and R² represents a hydrocarbon group with 6 or more and 24 or less carbons, and the total number of carbons in R¹ and R² is 18 or more and 30 or less; and M is a cation, excluding a hydrogen ion.

<2>

The softening base agent according to <1>, wherein each of the hydrocarbon groups of R¹ and R² in the formula 1 has 6 or more, preferably 8 or more and more preferably 10 or more, and 24 or less, preferably 20 or less and more preferably 17 or less carbons.

<3>

The softening base agent according to <1> or <2>, wherein at least one of R¹ and R² in the formula 1 is a hydrocarbon group having a branched structure or an unsaturated bond.

<4>

The softening base agent according to any one of <1> to <3>, wherein the total number of carbons in R¹ and R² in the formula 1 is 18 or more, preferably 20 or more, more preferably 21 or more and further preferably 22 or more, and 30 or less, preferably 28 or less, more preferably 26 or less and further preferably 25 or less.

<5>

The softening base agent according to any one of <1> to <4>, wherein the hydrocarbon groups of R¹ and R² in the formula 1 include a hydrocarbon group having a branched structure.

<6>

The softening base agent according to any one of <1> to <5>, wherein the hydrocarbon groups of R¹ and R² in the formula 1 include an unsaturated hydrocarbon group.

<7>

The softening base agent according to any one of <1> to <6>, wherein the hydrocarbon groups of R¹ and R² in the formula 1 include a saturated branched hydrocarbon group or an unsaturated linear hydrocarbon group.

<8>

The softening base agent according to any one of <1> to <7>, wherein R¹ and R² are hydrocarbon groups having the same structure.

<9>

The softening base agent according to any one of <1> to <7>, wherein R¹ and R² are hydrocarbon groups having different structures.

<10>

The softening base agent according to any one of <1> to <9>, wherein the hydrocarbon groups of R¹ and R² in the formula 1 include a hydrocarbon group having a branched structure, and the degree of branching that is defined by the following formula is 0.3 or less,

degree of branching=[(total number of terminal methyl groups in R¹ and R²)−2]/(total number of carbons in R¹ and R²)

<11>

The softening base agent according to any one of <1> to <9>, wherein the hydrocarbon groups of R¹ and R² in the formula 1 include a hydrocarbon group having a branched structure, and the degree of branching that is defined by the following formula is 0.2 or less,

degree of branching=[(total number of terminal methyl groups in R¹ and R²)−2]/(total number of carbons in R¹ and R²)

<12>

The softening base agent according to any one of <1> to <9>, wherein the hydrocarbon groups of R¹ and R² in the formula 1 include a hydrocarbon group having a branched structure, and the degree of branching that is defined by the following formula is 0.1 or less,

degree of branching=[(total number of terminal methyl groups in R¹ and R²)−2]/(total number of carbons in R¹ and R²)

<13>

The softening base agent according to any one of <1> to <12>, wherein the hydrocarbon groups of R¹ and R² in the formula 1 include a hydrocarbon group having a branched structure, and the degree of branching that is defined by the following formula is 0.01 or more,

degree of branching=[(total number of terminal methyl groups in R¹ and R²)−2]/(total number of carbons in R¹ and R²)

<14>

The softening base agent according to any one of <1> to <12>, wherein the hydrocarbon groups of R¹ and R² in the formula 1 include a hydrocarbon group having a branched structure, and the degree of branching that is defined by the following formula is 0.02 or more,

degree of branching=[(total number of terminal methyl groups in R¹ and R²)−2]/(total number of carbons in R¹ and R²)

<15>

The softening base agent according to any one of <1> to <12>, wherein the hydrocarbon groups of R¹ and R² in the formula 1 include a hydrocarbon group having a branched structure, and the degree of branching that is defined by the following formula is 0.04 or more,

degree of branching=[(total number of terminal methyl groups in R¹ and R²)−2]/(total number of carbons in R¹ and R²)

<16>

The softening base agent according to any one of <3>, <5>, <7> and <10> to <15> of <3> to <18>, wherein the hydrocarbon group having a branched structure is a hydrocarbon group having a branched chain in the second position, further a hydrocarbon group having a branched chain with 2 or more carbons in the second position, further an alkyl group having a branched chain with 2 or more carbons in the second position, further a hydrocarbon group having a branched chain derived from a Guerbet alcohol in the second position, and further a hydrocarbon group having a branched chain derived from a Guerbet alcohol only in the second position.

<17>

The softening base agent according to any one of <1> to <16>, wherein the hydrocarbon groups of R¹ and R² in the formula 1 include a hydrocarbon group having a branched structure, and the proportion of a hydrocarbon group having a branched structure at a carbon in the second or subsequent positions and only one methyl group bonded to the carbon in the second position, and further a hydrocarbon group represented by the following formula 2, is preferably 5 mol % or less, more preferably 4 mol % or less, further preferably 3 mol % or less, furthermore preferably 2 mol % or less, furthermore preferably 1 mol % or less and furthermore preferably 0 mol % in all the hydrocarbon groups of R¹ and R²,

—CH₂—CH(CH₃)—R²¹ Formula 2

wherein R²¹ is a hydrocarbon group with 3 or more and 21 or less carbons.

<18>

The softening base agent according to any one of <1> to <17>, wherein the hydrocarbon groups of R¹ and R² in the formula 1 include a hydrocarbon group having a branched structure, and the proportion of a hydrocarbon group having a branched structure only at a carbon in the third or subsequent positions and only one methyl group bonded to the carbon in the third position, and further a hydrocarbon group represented by the following formula 3, is preferably 10 mol % or less, more preferably 5 mol % or less, further preferably 2 mol % or less, furthermore preferably 1 mol % or less and furthermore preferably 0 mol % in all the hydrocarbon groups of R¹ and R²,

—R³¹—CH(CH₃)—R³² Formula 3

wherein R³¹ is a linear hydrocarbon group with 2 carbons, R³² is a hydrocarbon group, and the total number of carbons in R³¹ and R³² is 4 or more and 22 or less.

<19>

The softening base agent according to any one of <1> to <18>, wherein the hydrocarbon groups of R¹ and R² in the formula 1 include a hydrocarbon group having a branched structure, and the proportion of a hydrocarbon group having a branched structure only at a carbon in the fourth or subsequent positions and only one methyl group bonded to the carbon at the smallest branch position, and further a hydrocarbon group represented by the following formula 4, is preferably 50 mol % or less, more preferably 40 mol % or less, further preferably 30 mol % or less, furthermore preferably 20 mol % or less, furthermore preferably 10 mol % or less, furthermore preferably 5 mol % or less, furthermore preferably 1 mol % or less and furthermore preferably 0 mol % in all the hydrocarbon groups of R¹ and R²,

—R⁴¹—CH(CH₃)—R⁴² Formula 4

wherein R⁴¹ is a linear hydrocarbon group with 3 or more carbons, R⁴² is a hydrocarbon group, and the total number of carbons in R⁴¹ and R⁴² is 4 or more and 22 or less.

<20>

The softening base agent according to any one of <1> to <19>, wherein the agent contains a compound represented by the formula 1 in which R¹ and R² are hydrocarbon groups having the same structure, and a compound represented by the formula 1 in which R¹ and R² are hydrocarbon groups having different structures.

<21>

The softening base agent according to any one of <1> to <20>, wherein M in the formula 1 is a cation selected from an alkali metal ion and an alkanolammonium ion, preferably a cation selected from a sodium ion, a potassium ion, a triethanolammonium ion, a diethanolammonium ion and a monoethanolammonium ion, and more preferably a sodium ion.

<22>

The softening base agent according to any one of <1> to <21>, wherein, in results of measurement by 1³C-NMR under the conditions below, compound 1 has a proportion of the area of the region of signals in the range of 67.6 to 68 ppm relative to the area of the region of all signals between 60 and 69 ppm of preferably 5% or less, more preferably 4% or less, further preferably 3% or less, furthermore preferably 2% or less, furthermore preferably 1% or less and furthermore preferably 0%,

¹³C-NMR measurement conditions

Equipment: manufactured by Agilent Technologies, MR 400

Frequency: 400 MHz

Cumulative number: 1024

Latency time: 30 sec

Pulse angle: 45 deg

Deuterated solvent: CDCl₃

Sample concentration: 10%

Sample tube: 5 mmφ

<23>

The softening base agent according to any one of <1> to <22>, wherein, in results of the measurement by ¹³C-NMR under the conditions recited in <22>, compound 1 has a proportion of the area of the region of signals in the range of 60 to 61 ppm relative to the area of the region of all signals between 60 and 69 ppm of preferably 10% or less, more preferably 5% or less, further preferably 2% or less, furthermore preferably 1% or less and furthermore preferably 0%.

<24>

The softening base agent according to any one of <1> to <23>, wherein, in results of the measurement by ¹³C-NMR under the conditions recited in <22>, compound 1 has a proportion of the area of the region of signals in the range of 62 to 63.2 ppm relative to the area of the region of all signals between 60 and 69 ppm of preferably 50%. or less, more preferably 40% or less, further preferably 30% or less, furthermore preferably 20% or less, furthermore preferably 10% or less, furthermore preferably 5′, or less, furthermore preferably 1% or less and furthermore preferably 0%.

<25>

The softening base agent according to any one of <1> to <24>, wherein the agent is for use in fibers, further for use in fibers selected from natural fibers, synthetic fibers and semi-synthetic fibers, further for use in textile products, and further for use in textile products including the fibers.

<26>

The softening base agent according to <25>, wherein the fibers are fibers including cotton fibers.

<27>

The softening base agent according to <26>, wherein the content of cotton fibers in the fibers is preferably 5 mass % or more, more preferably 10 mass % or more, further preferably 15 mass % or more, furthermore preferably 20 mass % or more and furthermore preferably 100 mass %.

<28>

The softening base agent according to any one of <24> to <27>, wherein the textile products are textile products including cotton fibers.

<29>

The softening base agent according to <28>, wherein the content of cotton fibers in the textile products is preferably 5 mass % or more, more preferably 10 mass % or more, further preferably 15 mass % or more, furthermore preferably 20 mass % or more and furthermore preferably 100 mass %.

<30>

A fiber treatment agent composition containing the softening base agent according to any one of <1> to <29>.

<31>

The fiber treatment agent composition according to <30>, wherein the content of the compound represented by the formula 1 is 1 mass % or more and 90 mass % or less.

<32>

The fiber treatment agent composition according to <30> or <31>, wherein the content of the compound represented by the formula 1 is preferably 1 mass % or more, more preferably 2 mass, or more and further preferably 4 mass % or more, and preferably 90 mass % or less, more preferably 70 mass % or less and further preferably 50 mass % or less.

<33>

The fiber treatment agent composition according to any one of <30> to <32>, wherein the content of a softening base agent other than compound 1 (hereinafter also referred to as optional softening base agent) in the composition is less than 1 mass % and further less than 0.1 mass %.

<34>

The fiber treatment agent composition according to <33>, wherein the mass ratio of (content of optional softening base agent)/(content of compound 1) is 4.5 or less, further 4.0 or less, further 3.0 or less, further 2.0 or less, further 1.0 or less, further 0.50 or less, further 0.30 or less, further less than 0.10 and further 0.050 or less.

<35>

The fiber treatment agent composition according to <33> or <34>, wherein the optional softening base agent is a softening agent selected from a cationic softening agent and a nonionic softening base agent.

<36>

The fiber treatment agent composition according to any one of <30> to <35>, wherein the agent contains water, and is further a liquid composition containing water.

<37>

The fiber treatment agent composition according to <36>, wherein the agent contains water in an amount of the balance of the composition, and further in an amount that makes the total of the composition 100 mass %.

<38>

The fiber treatment agent composition according to <36> or <37>, wherein the proportion of compound 1 in the total of components other than water is 20 mass % or more, further 30 mass % or more, further 40 mass % or more, further 50 mass % or more, further 60 mass % or more, further 70 mass % or more, further 80 mass % or more, further 90 mass % or more, further 92 mass % or more and further 95 mass % or more, and 100 mass % or less.

<39>

The fiber treatment agent composition according to any one of <30> to <38>, wherein the pH at 20° C. is 4.0 or more, further 5.0 or more, further 5.5 or more, further 6.0 or more and further 7.0 or more.

<40>

A method for treating fibers including, treating the fibers with the softening base agent according to any one of <1> to <29>, wherein the compound represented by the formula 1 is used in an amount of 0.01% o.w.f. or more and 5% o.w.f. or less relative to the fibers, or the compound represented by the formula 1 is used in an amount of 0.05% o.w.f. or more and 5% o.w.f. or less relative to the fibers.

<41>

The method for treating fibers according to <40>, wherein the compound represented by the formula 1 is used in an amount of 0.01% o.w.f. or more, preferably 0.05% o.w.f. or more, more preferably 0.1% o.w.f. or more, further preferably 0.2% o.w.f. or more and furthermore preferably 0.3% o.w.f. or more, and 5% o.w.f. or less, preferably 4% o.w.f. or less, more preferably 3% o.w.f. or less and further preferably 2% o.w.f. or less relative to the fibers.

<42>

The method for treating fibers according to <40> or <41>, wherein the fibers are treated with a treatment liquid obtained by mixing the softening base agent with water having a hardness of 0° DH or more and 30° DH or less.

<43>

The method for treating fibers according to <42>, wherein the hardness of water is preferably 1° DH or more, more preferably 2° DH or more and further preferably 3° DH or more, and preferably 25° DH or less and more preferably 20° DH or less.

<44>

The method for treating fibers according to any one of <40> to <43>, wherein the fibers are fabric.

<45>

The method for treating fibers according to any one of <40> to <44>, wherein the fibers are treated in a washing process.

<46>

The method for treating fibers according to <45>, wherein the washing process is one or more treatments selected from washing, rinsing and dewatering the fibers.

<47>

Use of a compound represented by the following formula 1 as a softening base agent:

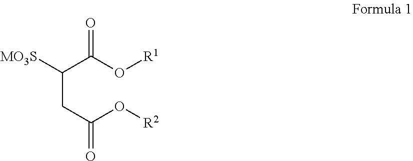

wherein each of R¹ and R² represents a hydrocarbon group with 6 or more and 24 or less carbons, and the total number of carbons in R¹ and R² is 18 or more and 30 or less; and M is a cation, excluding a hydrogen ion.

<48>

The use according to <47>, wherein at least one of R¹ and R² in the formula 1 is a hydrocarbon group having a branched structure or an unsaturated bond.

<49>

The use according to <47> or <48>, wherein the hydrocarbon group having a branched structure is a hydrocarbon group having a branched chain with 2 or more carbons in the second position.

### EXAMPLES

### Production Examples 1 to 8

A dialkyl sulfosuccinate of each production example listed in Table 1 was prepared as follows. In the reaction vessel shown in Table 1 equipped with a stirrer, a heating system, a distillation column and a nitrogen/vacuum connection, raw materials and the catalyst listed in Table 1 used to prepare a maleic acid diester were prepared in their respective amounts shown in Table 1, and after nitrogen substitution, reacted under nitrogen bubbling while dewatered at 100 to 130° C. until an acid value was lowered to an amount equivalent to that of p-toluenesulfonic acid. Subsequently, the catalyst was adsorbed to KYOWAAD® 500SH (manufactured by Kyowa Chemical Industry Co., Ltd.) in an amount of 1 mass relative to the total amount of the contents in the reaction vessel. After removing the adsorbent, an excess alcohol was removed by topping, thereby obtaining a maleic acid diester.

Next, in a 1-L glass reaction vessel, the maleic acid diester obtained above, sodium disulfite and ion exchange water were prepared in their respective amounts shown in Table 1, and using an alcohol polar solvent such as ethanol or the like to improve the compatibility of raw materials used to prepare each dialkyl sulfosuccinate listed in Table 1, reacted at 115° C. by a publicly-known method until it was confirmed by NMR that a double bond derived from the maleic acid diester disappeared. The reaction product was cooled to 50 to 65° C. and the remaining sodium hydrogen sulfite was oxidized with 30% hydrogen peroxide, and thereafter the pH was adjusted to 5 with 10% NaOH. The solvent and sodium sulfate were removed by distillation under reduced pressure, re-precipitation, liquid separation or the like, thereby obtaining each dialkyl sulfosuccinate listed in Table 1.

TABLE 1

| | | | | Production | | Production | | Production | | Production | | Production | |
|---|---|---|---|---|---|---|---|---|---|---|---|---|---|
| | | | | example 1 | | example 2 | | example 3 | | example 4 | | example 5 | |
| Dialkyl sulfosuccinate | | | | Di(2-propylheptyl) | | Di(2-butyloctyl)- | | Dodecyl/2- | | Octyl/cetyl- | | Dodecyl/3- | |
| | | | | sulfosuccinate | | sulfosuccinate | | butyloctyl- | | sulfosuccinate | | nonethyl- | |
| | | | | | | | | sulfosuccinate | | | | sulfosuccinate | |
| Preparation | Reaction | Type | | Four neck | | Four neck | | Four neck | | Four neck | | Four neck | |
| of maleic | vessel | | | flask | | flask | | flask | | flask | | flask | |
| acid diester | | Capacity | | 2 | L | 2 | L | 500 | mL | 500 | mL | 500 | mL |
| | Raw | Maleic | Preparation | 176.5 | g | 176.5 | g | 58.8 | g | 58.8 | g | 68.6 | g |
| | material | anhydride | amount (g) | | | | | | | | | | |
| | | | Preparation | 1.8 | mol | 1.8 | mol | 0.60 | mol | 0.60 | mol | 0.70 | mol |
| | | | amount (mol) | | | | | | | | | | |
| | | Alcohol 1 | Type | 2- | | 2- | | 2- | | Octanol | | Dodecanol | |
| | | | | propylheptanol | | bulytoctanol | | butyloctanol | | | | | |
| | | | Preparation | 626.6 | g | 737.8 | g | 123.0 | g | 86.0 | g | 143.5 | g |
| | | | amount (g) | | | | | | | | | | |
| | | | Preparation | 4.0 | mol | 4.0 | mol | 0.66 | mol | 0.66 | mol | 0.77 | mol |
| | | | amount (mol) | | | | | | | | | | |
| | | Alcohol 2 | Type | — | | — | | Dodecanol | | Cetanol | | Cis-3- | |
| | | | | | | | | | | | | nonene-1-ol | |
| | | | Preparation | — | | — | | 123.0 | g | 160.0 | g | 109.5 | g |
| | | | amount (g) | | | | | | | | | | |
| | | | Preparation | — | | — | | 0.66 | mol | 0.66 | mol | 0.77 | mol |
| | | | amount (mol) | | | | | | | | | | |
| | Catalyst | p-toluene- | Preparation | 2.5 | g | 2.5 | g | 0.82 | g | 0.82 | g | 0.84 | g |
| | | sulfonic | amount (mol) | | | | | | | | | | |
| | | acid mono- | Preparation | 0.013 | mol | 0.013 | mol | 0.0043 | mol | 0.0043 | mol | 0.0044 | mol |
| | | hydrate | amount (mol) | | | | | | | | | | |
| Preparation | Raw | Maleic acid | Preparation | 278 | g | 200 | g | 150 | g | 150 | g | 150 | g |
| of dialkyl | material | diester | amount (g) | | | | | | | | | | |
| sulfosuccinate | | | Preparation | 0.70 | mol | 0.44 | mol | 0.33 | mol | 0.31 | mol | 0.37 | mol |
| | | | amount (mol) | | | | | | | | | | |
| | | Sodium | Preparation | 73 | g | 46 | g | 35 | g | 32 | g | 38 | g |
| | | disulfite | amount (g) | | | | | | | | | | |
| | | | Preparation | 0.38 | mol | 0.24 | mol | 0.18 | mol | 0.17 | mol | 0.20 | mol |
| | | | amount (mol) | | | | | | | | | | |
| | | Ion exchange | Preparation | 48 | g | 18 | g | 13 | g | 12 | g | 14 | g |
| | | water | amount (g) | | | | | | | | | | |
| | | | Preparation | 2.7 | mol | 1.0 | mol | 0.73 | mol | 0.68 | mol | 0.81 | mol |
| | | | amount (mol) | | | | | | | | | | |

| | | | | Production | | Production | | Production | |
|---|---|---|---|---|---|---|---|---|---|
| | | | | example 6 | | example 7 | | example 8 | |
| Dialkyl sulfosuccinate | | | | Dicetyl | | Stearyl/oleyl- | | Cetyl/stearyl- | |
| | | | | sulfosuccinate | | sulfosuccinate | | sulfosuccinate | |
| Preparation | Reaction | Type | | Four neck | | Four neck | | Four neck | |
| of maleic | vessel | | | flask | | flask | | flask | |
| acid diester | | Capacity | | 2 | L | 500 | mL | 500 | mL |
| | Raw | Maleic | Preparation | 138 | g | 49.0 | g | 49.0 | g |
| | material | anhydride | amount (g) | | | | | | |
| | | | Preparation | 1.4 | mol | 0.50 | mol | 0.50 | mol |
| | | | amount (mol) | | | | | | |
| | | Alcohol 1 | Type | Cetanol | | Oleyl | | Cetanol | |
| | | | | | | alcohol | | | |
| | | | Preparation | 749 | g | 147.7 | g | 133.3 | g |
| | | | amount (g) | | | | | | |
| | | | Preparation | 3.1 | mol | 0.55 | mol | 0.55 | mol |
| | | | amount (mol) | | | | | | |
| | | Alcohol 2 | Type | — | | Stearyl | | Stearyl | |
| | | | | | | alcohol | | alcohol | |
| | | | Preparation | — | | 148.8 | g | 148.8 | g |
| | | | amount (g) | | | | | | |
| | | | Preparation | — | | 0.55 | mol | 0.55 | mol |
| | | | amount (mol) | | | | | | |
| | Catalyst | p-toluene | Preparation | 2.7 | g | 0.98 | g | 0.93 | g |
| | | sulfonic | amount (mol) | | | | | | |
| | | acid mono- | Preparation | 0.014 | mol | 0.0052 | mol | 0.0049 | mol |
| | | hydrate | amount (mol) | | | | | | |
| Preparation | Raw | Maleic acid | Preparation | 200 | g | 150 | g | 150 | g |
| of dialkyl | material | diester | amount (g) | | | | | | |
| sulfosuccinate | | | Preparation | 0.35 | mol | 0.24 | mol | 0.25 | mol |
| | | | amount (mol) | | | | | | |
| | | Sodium | Preparation | 37 | g | 25 | g | 26 | g |
| | | disulfite | amount (g) | | | | | | |
| | | | Preparation | 0.19 | mol | 0.13 | mol | 0.14 | mol |
| | | | amount (mol) | | | | | | |
| | | Ion exchange | Preparation | 18 | g | 9.6 | g | 10 | g |
| | | water | amount (g) | | | | | | |
| | | | Preparation | 1.0 | mol | 0.53 | mol | 0.56 | mol |
| | | | amount (mol) | | | | | | |

The components in Table 1 are listed below.

Maleic anhydride: manufactured by FUJIFILM Wako Pure Chemical Corporation, Wako Special Grade

2-propylheptanol: manufactured by FUJIFILM Wako Pure Chemical Corporation, Guaranteed Reagent

2-butyloctanol: 2-butyl-1-n-octanol, manufactured by FUJIFILM Wako Pure Chemical Corporation, Guaranteed Reagent

Octanol: manufactured by Kao Corporation, “KALCOL 0898”

Dodecanol: manufactured by Kao Corporation, “KALCOL 2098”

Cetanol: manufactured by Kao Corporation, “KALCOL 6098”

Oleyl alcohol: manufactured by Alfa Aesar

Cis-3-nonene-1-ol: manufactured by Tokyo Chemical Industry Co., Ltd.

Stearyl alcohol: manufactured by Kao Corporation, “KALCOL 8098”

P-toluenesulfonic acid monohydrate: manufactured by FUJIFILM Wako Pure Chemical Corporation, Guaranteed Reagent

Sodium disulfite: manufactured by FUJIFILM Wako Pure Chemical Corporation, Guaranteed Reagent

### Examples 1 to 5 and Comparative Examples 1 to 5

Using the dialkyl sulfosuccinates listed in Table 1 as softening base agents, softness and the stability of dispersions were evaluated in the following manner. The results are shown in Table 2. Note that Table 2 shows the structures in the formula 1 of the softening base agents. For compounds that do not qualify as compound 1, structures corresponding to those in the formula 1 are shown for convenience.

The softening base agents listed in Table 2 are as follows.
Inventive product 1: di(2-propylheptyl)-sulfosuccinate prepared in production example 1
Inventive product 2: di(2-butyloctyl)-sulfosuccinate prepared in production example 2
Inventive product 3: dodecyl/2-butyloctyl-sulfosuccinate prepared in production example 3
Inventive product 4: octyl/cetyl-sulfosuccinate prepared in production example 4
Inventive product 5: dodecyl/3-nonenyl-sulfosuccinate prepared in production example 5
Comparative product 1: di(2-ethylhexyl)sulfosuccinate, reagent, DIOCTYLSULFOSUCCINATE (MP Biomedical, Inc.)
Comparative product 2: dicetyl sulfosuccinate prepared in production example 6
Comparative product 3: stearyl/oleyl-sulfosuccinate prepared in production example 7
Comparative product 4: cetyl/stearyl-sulfosuccinate prepared in production example 8
Comparative product 5: α-olefin sulfonate, LIPOLAN PB-800CJ, manufactured by Lion Corporation

Method for Evaluating Softness

### 1) Pretreatment of Towel Used for Evaluation

Towels from which sizing agents or impurities were removed by the following pretreatment were used for evaluation.

In a fully automatic washing machine (manufactured by Panasonic Corporation, Model No.: NA-F60PB3), 52.22 g of a 10% diluent of a nonionic surfactant (manufactured by Kao Corporation, EMULGEN 108) was added as a detergent to 24 commercially available cotton towels (manufactured by Takei Towel Co., Ltd., TW220, white), and a series of washing processes (water volume 50 L, washing for 10 minutes→water-saving rinsing twice→dewatering for 9 minutes) were repeated 3 times using tap water of Wakayama city (the tap water is water having a hardness of 4° DH, and the same applies hereinafter) as water. Subsequently, the series of washing processes were repeated twice using only water. After that, the towels were left and naturally dried at room temperature (25° C.) for 24 hours.

### 2) Method for Treating Towel

In National MiniMini Washer NA-35, a predetermined amount of ion exchange water was placed (such that the bath ratio was 25 liters per kilogram of towels) and an aqueous calcium chloride solution (equivalent to 4000° DH) was added to make the hardness 20° DH, and while stirring them, a 5 mass % water dispersion of each softening base agent in Table 2 was added and stirred for 1 minute, and thereafter, 3 cotton towels (about 210 g in total) pretreated in the above 1) were placed therein and treated for 5 minutes under stirring. In this treatment, each softening base agent in Table 2 was used in an amount of 0.5% o.w.f. with reference to 3 cotton towels. Subsequently, the cotton towels were dewatered for 3 minutes in a dewatering tank of a two-tank washing machine (manufactured by TOSHIBA CORPORATION, Model No.: VH-52G(H)), and dried in a thermo-hygrostat at 23° C. and 40% RH for 24 hours. Similarly, an aqueous calcium chloride solution was added to make the hardness 8° DH and a 5 mass % water dispersion of each softening base agent was added in an amount of 0.3% o.w.f. to obtain a treatment liquid, and cotton towels were treated therewith and dried.

### 3) Softness Evaluation

A cotton towel treated with a formulation indicated in each score below was prepared as a reference by the methods in the above 1) and 2).

The softness of a cotton towel treated with each softening base agent listed in Table 2 was compared with that of each reference cotton towel to evaluate the softness. Scores (points) given by five panelists in accordance with the following criteria to make evaluations were averaged and listed in the table. Note that the scores given by the panelists to make evaluations could be decimal fraction values between values of two scores.

Score 1: as soft as that treated only with tap water at 20° C.

Score 2: as soft as that treated with a formulation using an indicator softening base agent in an amount of 0.025% o.w.f. in tap water at 20° C.

Score 3: as soft as that treated with a formulation using the indicator softening base agent in an amount of 0.050% o.w.f. in tap water at 20° C.

Score 4: as soft as that treated with a formulation using the indicator softening base agent in an amount of 0.075% o.w.f. in tap water at 20° C.

Score 5: as soft as that treated with a formulation using the indicator softening base agent in an amount of 0.100% o.w.f. in tap water at 20° C.

Here, an ester amide hydrochloride (2-[N-[3-alkanoyl(C14-20)aminopropyl]-N-methylamino]ethylalkano(C14-20)ate hydrochloride) was used as the indicator softening base agent.

Method for Evaluating Dispersion Stability

5 g of each softening base agent in Table 2 and 95 g of ion exchange water were mixed and stirred at 80° C. for 20 minutes, and thereafter stirred at room temperature (20° C.) for 20 minutes, and left at room temperature for 24 hours. After that, the mixture was further left at 5° C. for 24 hours. The dispersion stability was evaluated by observing the appearance of the mixture. Evaluation criteria for the dispersion stability were shown below. The dispersion stability was evaluated on the basis of the appearance observed at room temperature immediately after stirring at 80° C. for 20 minutes (immediately after preparation) and the appearance observed at a liquid temperature of 5° C. after leaving at 5° C. for 24 hours (after leaving at 5° C. for 24 hours).

*Evaluation Criteria for Dispersion Stability

Good: no precipitations

Average: a slight precipitation

Poor: a large amount of precipitation

TABLE 2

| | | | Example | | | | |
|---|---|---|---|---|---|---|---|
| | | | 1 | 2 | 3 | 4 | 5 |
| Softening | Compound | | Inventive | Inventive | Inventive | Inventive | Inventive |
| base agent | | | product 1 | product 2 | product 3 | product 4 | product 5 |
| | Structure in | R¹, R² | 2-propylheptyl | 2-butyloctyl | 2-butyloctyl | Octyl (linear | Dodecyl (linear |
| | formula 1 | | (branched | (branched | (branched | saturated):cetyl | saturated):3- |
| | | | saturated) | saturated) | saturated):dodecyl | (linear | nonenyl (linear |
| | | | | | (linear saturated) = | saturated) = | unsaturated) = |
| | | | | | 1:1 (molar ratio) | 1:1 (molar ratio) | 1:1 (molar ratio) |
| | | Total number of carbons in R¹ | 20 | 24 | 24 | 24 | 21 |
| | | and R² (molar average) | | | | | |
| | | M | Na | Na | Na | Na | Na |
| | | Degree of branching | 0.1 | 0.083 | 0.042 | 0 | 0 |

| | | | | | | |
|---|---|---|---|---|---|---|
| Dispersion | Immediately after | Good | Good | Good | Good | Good |
| stability | preparation | | | | | |
| | After leaving for 24 | Good | Good | Good | Average | Good |
| | hours at 5° C. | | | | | |
| Softness | 20° DH | 3.2 | 2.9 | 4.2 | 4.7 | 3.4 |
| (point) | 8° DH | 2.8 | 2.5 | 2.5 | 3.1 | 2.6 |

| | | | Comparative example | | | | |
|---|---|---|---|---|---|---|---|
| | | | 1 | 2 | 3 | 4 | 5 |
| Softening | Compound | | Comparative | Comparative | Comparative | Comparative | Comparative |
| base agent | | | product 1 | product 2 | product 3 | product 4 | product 5 |
| | Structure | R¹, R² | 2-ethylhexyl | Cetyl | Stearyl (linear | Cetyl (linear | — |
| | in formula 1 | | (branched | (linear | saturated):oleyl | saturated):stearyl | |
| | | | saturated) | saturated) | (linear unsaturated) = | (linear saturated) = | |
| | | | | | 1:1 (molar ratio) | 1:1 (molar ratio) | |
| | | Total number of carbons in R¹ | 16 | 32 | 36 | 34 | — |
| | | and R² (motor average) | | | | | |
| | | M | Na | Na | Na | Na | — |
| | | Degree of branching | 0.125 | 0 | 0 | 0 | — |

| | | | | | | |
|---|---|---|---|---|---|---|
| Dispersion | Immediately after | Good | Good | Poor | Good | Good |
| stability | preparation | | | | | |
| | After having for 24 | Good | Average | Poor | Poor | Good |
| | hours at 5° C. | | | | | |
| Softness | 20° DH | 1.7 | 2.3 | Not evaluable | 1.8 | 1.7 |
| (point) | 8° DH | 1.9 | 2.4 | 2.4 | 1.7 | 1.2 |

From the results in Table 2 showing that the inventive softening base agents impart higher softness than the comparative softening base agents, it can be seen that the inventive softening base agents are softening base agents superior to other anionic surfactants in fiber softening effect. Further, it can be seen that the inventive softening base agents exhibit more excellent softness when used with water having a higher hardness. In addition, it can be seen that the inventive softening base agents have excellent dispersion stability in water.

1. A softening base agent comprising a compound according to formula 1: 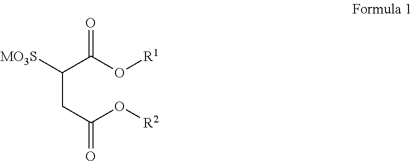
wherein:
each of R¹ and R² is independently a hydrocarbon group with 6 to 24 carbons;
a total number of carbons in R¹ and R² is 18 to 30; and
M is a cation, excluding a hydrogen ion.

2. The softening base agent according to claim 1, wherein at least one of R¹ and R² is a hydrocarbon group having a branched structure or an unsaturated bond.

3. The softening base agent according to claim 2, wherein:
at least one of R¹ and R² is a hydrocarbon group having a branched structure;
the hydrocarbon group having a branched structure has a branched chain in a second position; and
the branched chain is a hydrocarbon group with 2 or more carbons.

4. The softening base agent according to claim 1, wherein each of R¹ and R² is independently a hydrocarbon group with 6 to 17 carbons.

5. The softening base agent according to claim 1, wherein the agent comprises:
a first compound according to formula 1 in which R¹ and R² are hydrocarbon groups having the same structure; and
a second compound according to formula 1 in which R¹ and R² are hydrocarbon groups having different structures.

6. The softening base agent according to claim 1, wherein:
each of R¹ and R² independently comprises a hydrocarbon group having a branched structure; and
a proportion of hydrocarbon groups of R¹ and R² according to formula 4 among all hydrocarbon groups R¹ and R² in compounds according to formula 1 is 50 mol % or less:
—R⁴¹—CH(CH₃)—R⁴² Formula 4
wherein:
R⁴¹ is a linear hydrocarbon group with 3 or more carbons;
R⁴² is a hydrocarbon group; and
a total number of carbons in R⁴¹ and R⁴² is 4 to 22.

7. A fiber treatment agent composition comprising the softening base agent according to claim 1.

8. The fiber treatment agent composition according to claim 7, wherein a content of the compound according to formula 1 is 1 mass % to 90 mass % based on a total mass of the composition.

9. A method for treating fibers comprising, treating the fibers with the softening base agent according to claim 1, wherein the compound according to formula 1 is applied in an amount of 0.01% o.w.f. to 5% o.w.f relative to the fibers.

10. The method for treating fibers according to claim 9, wherein treating the fibers comprises treating with a treatment liquid obtained by mixing the softening base agent with water having a hardness of 0° DH to 30° DH.

11. The method for treating fibers according to claim 9, wherein the fibers are fabric.

12. The method for treating fibers according to claim 9, wherein treating the fibers comprises treating in a washing process.

13-15. (canceled)

16. The softening base agent according to claim 2, wherein:
at least one of R¹ and R² is a hydrocarbon group having a branched structure; and
the hydrocarbon group having a branched structure has a branched chain derived from a Guerbet alcohol in a second position.

17. The softening base agent according to claim 2, wherein:
at least one of R¹ and R² is a hydrocarbon group having a branched structure; and
the hydrocarbon group having a branched structure has a branched chain derived from a Guerbet alcohol only in a second position.

18. The softening base agent according to claim 1, wherein the total number of carbons in R¹ and R² is 20 to 25.

19. The fiber treatment agent composition according to claim 7, wherein a content of softening base agents other than the compound according to formula 1 in the composition is less than 1 mass %.

20. The fiber treatment agent composition according to claim 7, wherein a content of softening base agents other than the compound according to formula 1 in the composition is less than 0.1 mass %.

21. The fiber treatment agent composition according to claim 7, wherein a ratio of a mass of softening base agents other than the compound according to formula 1 to a mass of the compound of according to formula 1 in the composition is 4.5 or less.

22. The fiber treatment agent composition according to claim 7, wherein a ratio of a mass of softening base agents other than the compound according to formula 1 to a mass of the compound of according to formula 1 in the composition is 1.0 or less.

23. The method for treating fibers according to claim 9, wherein treating the fibers comprises treating with a treatment liquid obtained by mixing the softening base agent with water having a hardness of 1° DH to 25° DH.
